(19) United States
(12) Patent Application Publication
(10) Pub. No.: US 2024/0009250 A1
(43) Pub. Date: Jan. 11, 2024

(54) THERAPEUTIC APPLICATION OF CELL-FREE FAT EXTRACT TO ARTHRITIS

(71) Applicant: SHANGHAI SEME CELL TECHNOLOGY CO., LTD., Shanghai (CN)

(72) Inventors: Wenjie ZHANG, Shanghai (CN); Xifan HOU, Shanghai (CN)

(21) Appl. No.: 18/252,254

(22) PCT Filed: Oct. 21, 2021
(86) PCT No.: PCT/CN2021/125386

§ 371 Date: May 9, 2023

(30) Foreign Application Priority Data
Nov. 10, 2020 (CN) 202011248052.2

(51) Int. Cl. A61K 35/35; A61P 19/02; A61P 29/00

(52) U.S. Cl. CPC A61K 35/35; A61P 19/02; A61P 29/00

(57) ABSTRACT

A cell-free fat extract has a therapeutic applications. The cell-free fat extract is used for preparing a composition or preparation, and the composition or preparation is for one or more uses selected from the group consisting of: (i) prevention and/or treatment of arthritis; (ii) prevention and/or treatment of pain; and (iii) movement disorder.

### TECHNICAL FIELD

The present invention relates to the field of medicine, in particular, relates to the therapeutic application of cell-free fat extract to arthritis.

### BACKGROUND TECHNIQUE

Arthritis is an inflammatory disease that occurs in the joints and surrounding tissues of the human body and is caused by inflammation, infection, degradation, trauma, or other factors. The clinical manifestations of arthritis include redness, swelling, heat, pain, dysfunction of joint and joint deformities. In severe cases, it can lead to joint disability and affect the quality of life of patients.

Osteoarthritis (OA) is a type of arthritis that seriously affects the health of patients. Osteoarthritis is a degenerative disease of cartilage that begins with articular cartilage and gradually erodes into subchondral bone and surrounding tissues, leading to focal and erosive joint lesions, thereby causing joint pain, movement disorder, and deformity. The incidence of OA increases with age, and it has become the top disease that causes disability of middle-aged and elderly people, which seriously affects the work and life of patients. The current treatment methods include conservative treatment and surgical treatment. In the early stage of the disease process, intra-articular drug injections, such as sodium hyaluronate or glucocorticoids injection, are the mainstream conservative treatment methods for alleviating OA symptoms. However, there is still a lack of ideal treatment drugs, and finding safe and effective biological agents has become an urgent problem to be solved.

Therefore, it is necessary to develop a drug that can effectively treat arthritis in this field.

### SUMMARY OF THE INVENTION

The object of the present invention is to provide a use of a cell-free fat extract for preventing and/or treating arthritis.

In the first aspect of the present invention, it provides a use of a cell-free fat extract for the preparation of a composition or preparation, the composition or preparation is used for one or more uses selected from the group consisting of (i) preventing and/or treating arthritis; (ii) preventing and/or treating pain; (iii) movement disorder.

In another preferred embodiment, the arthritis includes osteoarthritis.

In another preferred embodiment, the arthritis includes drug-induced osteoarthritis.

In another preferred embodiment, the arthritis includes degenerative osteoarthritis.

In another preferred embodiment, the arthritis includes cartilaginous degenerative osteoarthritis.

In another preferred embodiment, the osteoarthritis includes arthritis at weight-bearing joints and joints which exercise more.

In another preferred embodiment, the osteoarthritis is selected from the group consisting of cervical osteoarthritis, lumbar osteoarthritis, knee osteoarthritis, hip osteoarthritis, and combinations thereof.

In another preferred embodiment, the osteoarthritis includes knee osteoarthritis. Knee osteoarthritis is a chronic osteoarthritis characterized by degenerative changes in the cartilage of the knee joints.

In another preferred embodiment, the pain includes arthritis pain.

In another preferred embodiment, the movement disorder includes the movement disorder caused by arthritis.

In another preferred embodiment, the prevention and/or treatment of arthritis is carried out through one or more means selected from the group consisting of:
(a) inhibiting fibrosis of articular cartilage; and/or
(b) improving the number of articular chondrocytes.

In another preferred embodiment, the improvement includes increase.

In another preferred embodiment, the cell-free fat extract is a cell-free fat extract obtained by extraction and preparation from fat in human or non-human mammals.

In another preferred embodiment, the non-human mammal is a monkey, an orangutan, a cow, a pig, a dog, a sheep, a rat or a rabbit.

In another preferred embodiment, the composition or preparation comprises pharmaceutical composition or preparation, food composition or preparation, nutraceutical composition or preparation, or dietary supplement.

In another preferred embodiment, the composition or preparation further comprises pharmaceutically, food, nutraceutical, or dietary acceptable carriers.

In another preferred embodiment, the dosage form of the composition or preparation is an oral preparation, an external preparation, or an injectable preparation.

In another preferred embodiment, the injectable preparation is an intravenous injection or an intramuscular injection.

In another preferred embodiment, the dosage form of the composition or preparation is a solid dosage form, a semi-solid dosage form, or a liquid dosage form, such as solution, gel, cream, lotion, ointment, cream, paste, cake, powder, patch, etc.

In another preferred embodiment, the dosage form of the composition or preparation is powder, granule, capsule, injection, tincture, oral solution, tablet or lozenge.

In another preferred embodiment, the composition or preparation is administered externally, topically, or by subcutaneous injection.

In another preferred embodiment, the cell-free fat extract contains no cell and no lipid droplet.

In another preferred embodiment, the lipid droplets are oil droplets released after fat cells are crushed.

In another preferred embodiment, the expression of “contain no lipid droplet” means that in the cell-free fat extract, the volume of oil droplets accounts for less than 1%, preferably less than 0.5%, more preferably less than 0.1% of the total liquid.

In another preferred embodiment, the cells are selected from the group consisting of endothelial cells, adipose stem cells, macrophages, and stromal cells.

In another preferred embodiment, the expression of “contain no cell” means that the average number of cells in 1 mL of the cell-free fat extract is ≤1, preferably ≤0.5, more preferably ≤0.1, or 0.

In another preferred embodiment, the cell-free fat extract is a naturally-obtained nano-fat extract without added ingredients.

In another preferred embodiment, the expression of “without added ingredients” means that no solution, solvent, small molecule, chemical preparation, and biological additive are added during the preparation of the fat extract except rinsing step.

In another preferred embodiment, the cell-free fat extract is prepared by centrifuging the fatty tissue after emulsification.

In another preferred embodiment, the cell-free fat extract contains one or more components selected from the group consisting of IGF-1, BDNF, GDNF, TGF-β1, HGF, bFGF, VEGF, TGF-β1, PDGF, EGF, NT-3, GH, G-CSF, and the combinations thereof.

In another preferred embodiment, the cell-free fat extract contains (but is not limited to) one or more components selected from the group consisting of IGF-1, BDNF, GDNF, bFGF, VEGF, TGF-β1, HGF, PDGF, and the combinations thereof.

In another preferred embodiment, the cell-free fat extract is cell-free fat extract liquid.

In another preferred embodiment, in the cell-free fat extract, the concentration of IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of IGF-1 to VEGF is 20-100:1, preferably 30-more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20:1, preferably 4-15:1, more preferably 6-12:1, and most preferably 8.5-9.5:1.

In another preferred embodiment, the weight ratio of bFGF to VEGF is 0.2-8:1, preferably 0.5 5:1-more preferably 0.6-2:1, more preferably 0.8-1.6:1, and most preferably 1-1.5:1.

In another preferred embodiment, the weight ratio of TGF-β1 to VEGF is 1-20:1, preferably 1-more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3:1, preferably 0.2-2:1, more preferably 0.4-1.5:1, and most preferably 0.7-1.2:1.

In another preferred embodiment, the cell-free fat extract is prepared by the following method:
(1) providing a fat tissue raw material, shredding the fat tissue raw material, and rinsing (e.g., with physiological saline), thereby obtaining a rinsed fat tissue;
(2) centrifuging the rinsed fat tissue to obtain a layered mixture;
(3) discharging the oil at the upper layer and the liquid at the bottom layer from the layered mixture and collecting the intermediate layer (that is, the fat layer containing fat cells);
(4) subjecting the intermediate layer to emulsification to obtain a emulsified fat mixture (also called nano-fat);
(5) centrifuging the emulsified fat mixture to obtain an intermediate liquid layer, which is a primary fat extract; and
(6) subjecting the primary fat extract to filtration and sterilization to obtain the cell-free fat extract.

In the second aspect of the present invention, it provides a method for preparing a cell-free fat extract, the method comprises the steps:
(1) providing a fat tissue raw material, shredding the fat tissue raw material, and rinsing (e.g., with physiological saline), thereby obtaining a rinsed fat tissue;
(2) centrifuging the rinsed fat tissue to obtain a layered mixture;
(3) discharging the oil at the upper layer and the liquid at the bottom layer from the layered mixture and collecting the intermediate layer (that is, the fat layer containing fat cells);
(4) subjecting the intermediate layer to emulsification to obtain a emulsified fat mixture (also called nano-fat);
(5) centrifuging the emulsified fat mixture to obtain an intermediate liquid layer, which is a primary fat extract; and
(6) subjecting the primary fat extract to filtration and sterilization to obtain the cell-free fat extract.

In another preferred embodiment, the cell-free fat extract is described as the first aspect of the present invention.

In another preferred embodiment, in the step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, the centrifugation temperature is 2-6° C.

In another preferred embodiment, in the step (4), the emulsification is mechanical emulsification.

In another preferred embodiment, the mechanical emulsification is performed by repeated blowing by a syringe (e.g., blowing 20-200 times, preferably 20-150 times, more preferably 20-100 times, more preferably 30-50 times).

In another preferred embodiment, the blowing method is that two 10 ml injection syringes are connected to a three-way pipe and repeatedly push back and forth at a constant speed.

In another preferred embodiment, in the step (4), the emulsification is by means of crushing through a tissue homogenizer.

In another preferred embodiment, the step (5) further includes freezing and thawing the emulsified fat mixture before centrifuging the emulsified fat mixture.

In another preferred embodiment, the thawed mixture is used for centrifugation after freezing and thawing treatment.

In another preferred embodiment, the freezing temperature is from −50° C. to −120° C., preferably from −60° C. to −100° C., more preferably from −70° C. to −90° C.

In another preferred embodiment, the thawing temperature is 20-40° C., preferably 25-40° C., more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fat tissue layer, the third layer is a liquid layer (i.e., an intermediate liquid layer), and the fourth layer is a cell/tissue debris precipitation layer.

In another preferred embodiment, in the step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, the centrifugation temperature is 2-6° C.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filtration can remove fat cells from the primary fat extract.

In another preferred embodiment, in the step (6), the filtering and sterilization are carried out through a filter (such as a 0.22 μm microporous filter membrane).

In another preferred embodiment, the filter is a microporous membrane filter.

In another preferred embodiment, the pore size of the microporous membrane is 0.05-0.8 μm, preferably 0.1-0.5 μm, more preferably 0.1-0.4 μm, more preferably 0.15-0.3 μm, more preferably 0.2-0.25 μm, and most preferably 0.22 μm.

In another preferred embodiment, in the step (6), the filtering and sterilization is carried out by first filtering through a first filter that can filter cells, and then through a second filter (such as a 0.22 μm filter) that can filter pathogens (such as bacteria).

In another preferred embodiment, the step (6) further includes subpackaging the fat extract to obtain a subpackaged product. (The subpacked extract may be stored at −20° C. for later use; it may be used directly after thawing at low temperature (e.g. −4° C.) or at normal temperature, or stored at low temperature (e.g. 4° C.) for a period of time after thawing for later use).

In the third aspect of the present invention, it provides a cell-free fat extract, which is prepared by a method as described in the second aspect of the present invention.

In the fourth aspect of the present invention, it provides a composition or preparation comprising (a) a cell-free fat extract as described in the third aspect of the present invention; and (b) pharmaceutically, food, nutraceutical, or dietary acceptable carriers or excipients.

In another preferred embodiment, the composition is pharmaceutical composition, food composition, health product composition, or dietary supplement.

In another preferred embodiment, the dosage form of the composition or preparation is oral preparation, external preparation, or injection preparation.

In another preferred embodiment, the dosage form of the composition or preparation is powder, granule, capsule, injection, tincture, oral solution, tablet or lozenge.

In another preferred embodiment, the injection is intravenous injection or intramuscular injection.

In another preferred embodiment, the dosage form of the composition or preparation is solid dosage form, semi-solid dosage form, or liquid dosage form, such as solution, gel, cream, lotion, ointment, cream, paste, cake, powder, patch.

In another preferred embodiment, in the composition or preparation, the mass percentage of the cell-free fat extract is 5 wt %, preferably 1-20 wt %, based on the total weight of the composition or preparation.

In the fifth aspect of the present invention, it provides a method for preparing a composition or preparation as described in the fourth aspect of the present invention, wherein the method comprises the steps of mixing a cell-free fat extract as described in the third aspect of the present invention with pharmaceutically, food, nutraceutical, or dietary acceptable carriers or excipients to form a composition or preparation.

In the sixth aspect of the present invention, it provides a method for (i) preventing and/or treating arthritis; (ii) preventing and/or treating pain; (iii) movement disorder, comprising administering a cell-free fat extract as described in the third aspect of the present invention to a subject in need thereof.

In another preferred embodiment, the subject is human or non-human mammal.

In another preferred embodiment, the non-human mammal includes rodent, such as a rat and a mouse.

It should be understood that, within the scope of the present invention, the above technical features of the present invention and the technical features specifically described in the following descriptions (such as the examples) can be combined with each other to form a new or preferred technical solution. Due to space limitations, they will not be repeated herein.

### DESCRIPTION OF THE DRAWINGS

FIG. 1 shows the changes in animal weight and time in each group after dosing.

FIG. 2 shows the pressure value of claw removal in rats before and after modeling.

FIG. 3 shows the pressure value of claw removal of rats in each group before and after dosing.

FIG. 4 shows absolute value of bipedal pressure difference in rats before and after modeling.

FIG. 5 shows absolute value of bipedal pressure difference of rats in each group before and after dosing.

FIGS. 6A-6E show the HE staining results (200×) of different groups of rats.

FIGS. 7A-7E show the Safranin O-fast green staining results (200×) of different groups of rats.

### DETAILED DESCRIPTION OF THE INVENTION

After extensive and thorough research, the present inventors have developed a cell-free fat extract showing excellent therapeutic effects on arthritis and its symptoms of pain and movement disorder for the first time. The present invention has been completed on this basis.

### Terms

Unless otherwise defined, all technical and scientific terms used herein have the same meaning as commonly understood by one of ordinary skill in the art to which this invention belongs.

As used herein, the terms “include” “contain” and “comprise” are used interchangeably and include not only open-ended definition, but also semi-closed definition, and closed definition. In other words, the terms include “consisting of”, “substantially consisting of”.

As used herein, the terms “cell-free fat extract” “cell free fat extract” and “CEFFE” are used interchangeably.

In the present invention, the term “prevent” means a method of preventing the onset of a disease and/or its accompanying symptoms or protecting a subject from developing a disease. The “prevent” used herein also includes delaying the onset of a disease and/or its accompanying symptoms and reducing the risk of illness in a subject.

“Treat” described in the present invention includes delaying and terminating the progression of a disease, or eliminating a disease. 100% inhibition, elimination, and reversal are not required. In some embodiments, compared to the level observed in the absence of composition, kit, food kit or health care kit, active ingredient combination described herein, the composition or pharmaceutical composition of the present invention reduces, inhibits and/or reverses diabetes, for example, by at least about 10%, at least about 30%, at least about 50%, or at least about 80%.

As used herein, “improve” includes prevent, treat, relieve, reverse and alleviate, etc.

As used herein, “IGF-1” refers to insulin-like growth factors-β1.

As used herein, “BDNF” refers to brain-derived neurotrophic factor.

As used herein, “GDNF” refers to glial cellline-derived neurotrophic factor.

As used herein, “bFGF” refers to basic fibroblast growth factor.

As used herein, “VEGF” refers to vascular endothelial growth factor.

As used herein, “TGF-β1” refers to transforming growth factor-β1.

As used herein, “HGF” refers to hepatocyte growth factor.

As used herein, “PDGF” refers to platelet derived growth factor.

As used herein, “EGF” refers to epidermal growth factor.

As used herein, “NT-3” refers to neurotrophins-3.

As used herein, “GH” refers to growth hormone.

As used herein, “G-CSF” refers to granulocyte colony stimulating factor.

Cell-Free Fat Extract (CEFFE) and the Preparation Method Therefor

As used herein, the term “cell-free fat extract of the present invention”, “extract of the present invention”, and “fat extract of the present invention”, etc. are used interchangeably and refer to an adipose tissue-derived extract (or extract liquid) prepared without adding any solution, solvent, small molecule, chemical preparation, and biological additive during the preparation of the fat extract (except for the rinsing step). An exemplary method for preparing the extract of the present invention is as described in the second aspect of the present invention above. In addition, it should be understood that although it is not necessary to add any additives (or added ingredients) during the preparation process of the extract of the present invention, some or small amount of safe substance (such as small amount of water) that do not negatively or adversely affect the activity of the extract herein may also be added.

In another preferred embodiment of the present invention, the cell-free fat extract is cell-free fat extract liquid.

The cell-free fat extract described in the present invention may include a variety of cytokines. Representatively, the cell-free fat extract comprises one or more of IGF-1, BDNF, GDNF, TGF-β, HGF, bFGF, VEGF, TGF-β1, PDGF, EGF, NT-3, GH, G-CSF.

In another preferred embodiment, in the cell-free fat extract, the concentration of IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20:1, preferably 4-15:1, more preferably 6-12:1, and most preferably 8.5-9.5:1.

In another preferred embodiment, the weight ratio of bFGF to VEGF is 0.2-8:1, preferably 0.5 5:1-more preferably 0.6-2:1, more preferably 0.8-1.6:1, and most preferably 1-1.5:1.

In another preferred embodiment, the weight ratio of TGF-β1 to VEGF is 1-20:1, preferably 1-more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3:1, preferably 0.2-2:1, more preferably 0.4-1.5:1, and most preferably 0.7-1.2:1.

Preferably, the cell-free fat extract described in the present invention is obtained by a method as described above in the second aspect of the present invention.

Representatively, the cell-free fat extract described in the present invention is prepared by the following method:
(1) providing a fat tissue raw material, shredding the fat tissue raw material, and rinsing (e.g., with physiological saline), thereby obtaining a rinsed fat tissue;
(2) centrifuging the rinsed fat tissue to obtain a layered mixture;
(3) discharging the oil at the upper layer and the liquid at the bottom layer from the layered mixture and collecting the intermediate layer (that is, the fat layer containing fat cells);
(4) subjecting the intermediate layer to emulsification to obtain a emulsified fat mixture (also called nano-fat);
(5) centrifuging the emulsified fat mixture to obtain an intermediate liquid layer, which is a primary fat extract; and
(6) subjecting the primary fat extract to filtration and sterilization to obtain the cell-free fat extract.

In another preferred embodiment, the cell-free fat extract is described as the first aspect of the present invention.

In another preferred embodiment, in the step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, in the step (4), the emulsification is mechanical emulsification.

In another preferred embodiment, the mechanical emulsification is performed by repeated blowing by a syringe (e.g., blowing 20-200 times, preferably 20-150 times, more preferably 20-100 times, more preferably 30-50 times).

In another preferred embodiment, the blowing method is that two 10 ml injection syringes are connected to a three-way pipe and repeatedly push back and forth at a constant speed.

In another preferred embodiment, in the step (4), the emulsification is by means of crushing through a tissue homogenizer.

In another preferred embodiment, the step (5) further includes freezing and thawing the emulsified fat mixture before centrifuging the emulsified fat mixture.

In another preferred embodiment, the thawed mixture is used for centrifugation after freezing and thawing treatment.

In another preferred embodiment, the freezing temperature is from −50° C. to −120° C., preferably from −60° C. to −100° C., more preferably from −70° C. to −90° C.

In another preferred embodiment, the thawing temperature is 20-40° C., preferably 25-40° C., more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fat tissue layer, the third layer is a liquid layer (i.e., an intermediate liquid layer), and the fourth layer is a cell/tissue debris precipitation layer.

In another preferred embodiment, in the step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filtration can remove fat cells from the primary fat extract.

In another preferred embodiment, in the step (6), the filtering and sterilization are carried out through a filter (such as a 0.22 μm microporous filter membrane).

In another preferred embodiment, the filter is a microporous membrane filter.

In another preferred embodiment, the pore size of the microporous membrane is 0.05-0.8 μm, preferably 0.1-0.5 μm, more preferably 0.1-0.4 μm, more preferably 0.15-0.3 μm, more preferably 0.2-0.25 μm, and most preferably 0.22 μm.

In another preferred embodiment, in the step (6), the filtering and sterilization is carried out by first filtering through a first filter that can filter cells, and then through a second filter (such as a 0.22 μm filter) that can filter pathogens (such as bacteria).

In another preferred embodiment, the step (6) further includes subpackaging the fat extract to obtain a subpackaged product. (The subpacked extract may be stored at −20° C. for later use; it may be used directly after thawing at low temperature (e.g. −4° C.) or at normal temperature, or stored at low temperature (e.g. 4° C.) for a period of time after thawing for later use).

Arthritis

Arthritis refers to an inflammatory disease that occurs in the joints and surrounding tissues of the human body and is caused by inflammation, infection, degradation, trauma, or other factors. The clinical manifestations of arthritis include redness, swelling, heat, pain, dysfunction of joint, and joint deformities. In severe cases, it can lead to joint disability and affect the quality of life of patients.

Representative, the arthritis described in the present invention is osteoarthritis.

Osteoarthritis and its Symptoms

Osteoarthritis (OA) is a degenerative disease of cartilage that begins with articular cartilage and gradually erodes to the subchondral bone and surrounding tissues, leading to focal and erosive joint lesions, thereby causing symptoms such as joint pain, stiffness, swelling, movement disorder, and deformity.

In the present invention, there are many inducing factors for osteoarthritis, and the etiology is still fully understood, which may be related to factors such as advanced age, obesity, medication, and occupational overuse.

In the present invention, there is no specific limitation on the location of occurrence of osteoarthritis, for example, it can be weight-bearing joints and joints which exercise more, such as arthritis in cervical spine, lumbar spine, knee joint, hip joint, and other parts.

Compositions and Administration

The compositions described herein include (but are not limited to) pharmaceutical compositions, food compositions, health care compositions, and dietary supplements, etc.

Representatively, the cell-free fat extract of the present invention may be prepared as pharmaceutical compositions in the dosage forms such as tablet, capsule, powder, microgranule, solution, lozenge, jelly, cream formulations, spirit, suspension, tincture, poultice, liniment, lotion, and aerosol. Pharmaceutical compositions can be prepared by commonly known preparation techniques, and suitable pharmaceutical additives can be added to the drug.

The compositions of the present invention may also include pharmaceutically, food, nutraceutical, or dietary acceptable carriers. “Pharmaceutically, food, nutraceutical, or dietary acceptable carrier” means one or more compatible solids or liquid fillers or gelatinous materials which are suitable for human use and should be of sufficient purity and sufficiently low toxicity. “Compatible” herein means that each component in the composition can be admixed with the compounds of the present invention and with each other without significantly reducing the efficacy of the compounds. Some examples of pharmaceutically, food, nutraceutical, or dietary acceptable carriers include cellulose and the derivatives thereof (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agent (such as sodium dodecyl sulfate), coloring agents, flavoring agents, stabilizers, antioxidants, preservatives, pyrogen-free water, etc.

There is no special limitation of administration mode for the compositions of the present invention, and the representative administration mode includes (but is not limited to) oral administration, parenteral (intravenous or intramuscular) administration, or topical administration, preferably oral administration and injection administration.

The dosage form of the composition or preparation described herein is oral preparation, external preparation, or injection preparation. Representatively, solid dosage forms for oral administration or dosing include capsules, tablets, pills, powders and granules. In these solid dosage forms, the active compounds are mixed with at least one conventional inert excipient (or carrier), such as sodium citrate or dicalcium phosphate, or mixed with any of the following components: (a) fillers or compatibilizer, for example, starch, lactose, sucrose, glucose, mannitol and silicic acid; (b) binders, for example, hydroxymethyl cellulose, alginates, gelatin, polyvinylpyrrolidone, sucrose and arabic gum; (c) humectant, such as glycerol; (d) disintegrating agents, for example, agar, calcium carbonate, potato starch or tapioca starch, alginic acid, certain composite silicates, and sodium carbonate; (e) dissolution-retarding agents, such as paraffin; (f) absorption accelerators, for example, quaternary ammonium compounds; (g) wetting agents, such as cetyl alcohol and glyceryl monostearate; (h) adsorbents, for example, kaolin; and (i) lubricants such as talc, calcium stearate, magnesium stearate, solid polyethylene glycol, sodium lauryl sulfate, or the mixtures thereof. In capsules, tablets and pills, the dosage forms may also contain buffering agents.

The solid dosage forms such as tablets, sugar pills, capsules, pills and granules can be prepared by using coating and shell materials, such as enteric coatings and any other materials known in the art. They can contain opaque agents.

Liquid dosage forms for oral administration or dosing include pharmaceutically acceptable emulsions, solutions, suspensions, syrups or tinctures. In addition to the active compounds, the liquid dosage forms may contain conventional inert diluents known in the art such as water or other solvents, solubilizers and emulsifiers, for example, ethanol, isopropanol, ethyl carbonate, ethyl acetate, propylene glycol, 1,3-butanediol, dimethyl formamide, as well as oil, in particular, cottonseed oil, peanut oil, corn germ oil, olive oil, castor oil and sesame oil, or the combinations thereof.

Besides these inert diluents, the composition may also contain additives such as wetting agents, emulsifiers, and suspending agents, sweeteners, flavoring agents and perfumes.

In addition to the active component, the suspension may contain suspending agent, for example, ethoxylated isooctadecanol, polyoxyethylene sorbitol and sorbitan esters, microcrystalline cellulose, methanol aluminum and agar, or the combinations thereof.

The compositions for parenteral injection may comprise physiologically acceptable sterile aqueous or anhydrous solutions, dispersions, suspensions or emulsions, and sterile powders which can be re-dissolved into sterile injectable solutions or dispersions. Suitable aqueous and non-aqueous carriers, diluents, solvents or excipients include water, ethanol, polyols and any suitable mixtures thereof.

The dosage forms of the compounds of the invention for topical administration include ointment, powder, patch, spray, and inhalant. The active ingredient is mixed under sterile conditions with physiologically acceptable carriers and any preservatives, buffers, or propellants that may be required if necessary.

The cell-free fat extract of the present invention can be administered alone or in combination with other drugs for preventing and/or treating fatty liver and/or its complications.

When the compositions are administered, a safe and effective amount of cell-free fat extract of the present invention is administered to a human or non-human mammal (such as rat, mouse, dog, cat, cow, chicken, duck, etc.) in need thereof, wherein the dose of administration is an effective administration dosage which is considered pharmaceutically, food, or nutraceutical acceptable. As used herein, the term “safe and effective amount” refers to an amount that is functional or active in humans and/or animals and is acceptable to humans and/or animals. It should be understood by those of ordinary skill in the art that the “safe and effective amount” may vary depending on the form of the pharmaceutical composition, the route of administration, the excipients of the drug used, the severity of the disease, and the combination with other drugs. For example, for a person weighed 60 kg, the daily dose is usually 0.1 to 1000 mg, preferably 1 to 600 mg, more preferably 2 to 300 mg. Of course, the specific dose should also take into account the route of administration, patient healthy status and other factors, which are within the skill of an experienced physician.

The Main Advantages of the Present Invention Include:

The present invention discovered the cell-free fat extract shows excellent therapeutic effects on arthritis and its symptoms of pain and movement disorder for the first time.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacturer's instructions. Unless indicated otherwise, percentage and parts are calculated by weight.

### Example 1

### 1. Experimental Method

1.1. Preparation of Cell-Free Fat Extract (CEFFE)

The fat was obtained from volunteers with informed consent. The preparation method of cell-free fat tissue extract is as follows:
(1) Fatty tissue was obtained from 6 healthy women who underwent conventional liposuction, with an average age of 31 years (24-36 years). After anesthesia with local injection of swelling solution, a 3 mm liposuction cannula with a large lateral hole (2 mm×7 mm) connected to a 20 mL syringe was used, the fat was manually aspirated radially under negative pressure, then the obtained fat was left upright and stationary, rinsed 3 times with saline after removal of the swelling solution.
(2) The rinsed fatty tissue was taken, placed in a centrifuge tube, then placed in a centrifuge and centrifuged at 1200 g for 3 minutes at 4° C. to obtain a layered mixture.
(3) For the layered mixture, the oil at the upper layer and the liquid at the bottom layer were discarded and the intermediate layer (i.e. the fat layer containing fat cells) was collected.
(4) The intermediate layer were pushed back and forth repeatedly and uniformly for 30 times with two 10 ml syringes connected to a three-way tube, performing mechanical emulsification, thereby obtaining a mechanically emulsified fat mixture (also called nano-fat).
(5) The mechanically emulsified fat mixture was placed into a −80° C. refrigerator for freezing, and then thawed in a 37° C. water bath. After a single freeze-thaw cycle, the thawed fat mixture was centrifuged at 1200 g for 5 minutes at 4° C. to obtain a layered mixture, which was separated into 4 layers, wherein the first layer was the oil layer, the second layer was the residual fatty tissue layer, the third layer was the liquid layer, and the fourth layer was the cell/tissue debris precipitation layer. The oil layer and the residual fatty tissue layer were discarded and the liquid layer was aspirated, avoiding contamination of the cellular/tissue debris precipitation layer during the aspiration process, thereby resulting in a primary fat extract.
(6) The obtained primary fat extract was filtered and degermed through a 0.22 μm filter, thereby sterilizing and removing any live cells that may have been mixed, resulting in a cell-free fat extract (CEFFE) that was subpackaged, stored and frozen at −20° C., then thawed at 4° C. when used.

The cytokine content of the prepared cell-free fat extract was detected using ELISA immunosorbent assay kits, including cytokines such as IGF-1, BDNF, GDNF, bFGF, VEGF, TGF-β1, HGF and PDGF. The average concentrations of 6 samples were as follows: IGF-1 (9840.6 pg/ml), BDNF (1764.5 pg/ml), GDNF (1831.9 pg/ml), bFGF (242.3 pg/ml), VEGF (202.9 pg/ml), TGF-β1 (954.5 pg/ml), HGF (898.4 pg/ml), and PDGF (179.9 pg/ml).

1.2 Establishment of a Rat Osteoarthritis (OA) Model, Grouping, and Administration

Sodium iodoacetate (MIA) is one of the common compounds that induce osteoarthritis (OA) model. Its mechanism is to promote the production of reactive oxygen species, induce depolarization of mitochondrial membrane, further increase the release of cytochrome C, activate the activity of Caspase3, and lead to apoptosis of chondrocytes.

This study used MIA knee joint injection to establish a model, the experimental animals were 8-week-old male SD rats. 6 rats were randomly selected as the normal control group, while the other rats were used for OA modeling. After model rats were anesthetized with isoflurane, they were injected with 50 μL of 40 mg/mL MIA solution into the joint cavity of the left hind limb. One week after modeling, 24 OA rats with significantly increased bipedal pressure difference and significantly decreased Von Frey value in the left hind foot were selected and randomly divided into 4 groups, with 6 rats in each group. The administration dosage for rats in the normal control group and rats after modeling was as follows:

TABLE 1
Administration Dose of Different Groups of Rats

| | | administration capacity |
|---|---|---|
| Group | | (μL/side) |
| Group 1 | Normal control group | −/− |
| Group 2 | Model control group | 60 |
| Group 3 | CEFFE low dose group | 15 |
| Group 4 | CEFFE medium dose group | 30 |
| Group 5 | CEFFE high dose group | 60 |

Note: rats in the normal control group were not subjected to any treatment throughout the entire process; rats in the model control group were administrated by 0.9% sodium chloride injection, while rats in the CEFFE low dose group, CEFFE medium dose group, and CEFFE high dose group were administrated different doses of cell-free fat extract (CEFFE); the rats in the model control group, CEFFE low dose group, CEFFE medium dose group, and CEFFE high dose group were administered intravenously to the left joint once every 2 weeks (a total of 4 doses), with the day of administration being Day 1 (counted as the first week).

1.3 Von Frey Testing

Detection indicator: value of claw removal (g).

Testing time point: once before modeling, 1 week after modeling, and 1 week after each administration.

Detection method: The animal were placed in a testing container and adapted to the environment for about 10 minutes. The left paw retraction pressure of the animal was detected (when the pressure reached the maximum value of 50 g, and the incubation period lasted for 40 seconds, the animal still did not show retraction response, the experiment was terminated manually to avoid tissue damage, the retraction incubation period and pressure value were recorded as 40 s and 50 g respectively). Each paw was detected for 2-3 times, with an interval of 1-3 minutes. The two data with similar results was taken to calculate the average value. (If the measurement process was stopped due to spontaneous animal activity, re-measurement was required after an interval of 1-3 minutes. If unsure, the average value can be measured multiple times to ensure that the animal's paw retraction was caused by mechanical stimulation.)

Instrument used: Dynamic plantar tactile sensor (model: 37450; manufacturer: Ugo Basile).

1.4 Measurement of Bipedal Balance

Detection indicator: bipedal pressure difference (g).

Testing time point: once before modeling, 1 week after modeling, and 1 week after each administration.

Detection method: The animal were placed in a container. When the animal calmed down, the left and right feet were in the corresponding sensing area and the number displaying the weight of the left and right feet remained relatively stable (constant for at least 3 seconds), the data was recorded.

Instrument used: Weight bearing asymmetry (model: 600MR; manufacturer: IITC life science).

1.5 Pathological Examination

Animals were euthanized one week after the end of the last administration and behavioral testing. The left joint tissue was rinsed with physiological saline, fixed in 10% neutral formalin solution, decalcified and dehydrated, and sliced (cross cut 2 sections along the cartilage surface, 5 μM/section), one section was stained with HE and the other section was stained with Safranin O-fast green staining.

The conventional 4-grade method was used in HE staining to grade the microscopic results, which are slight (+), mild (++), moderate (+++), and severe (++++), respectively, to facilitate intergroup comparison. Safranin O-fast green staining method: 3 visual fields were randomly selected from each section for scoring. The scoring criteria were shown in Table 2:

TABLE 2
Scoring Criteria for Safranin O-Fast Green Staining Method
Safranin O-fast green staining

| | |
|---|---|
| 0 | Uniform staining of articular cartilage |
| 1 | Loss of superficial staining of hyaline cartilage <50% |
| 2 | Loss of superficial staining of hyaline cartilage >50% |
| 3 | Loss of staining of upper ⅔ hyaline cartilage <50% |
| 4 | Loss of staining of upper ⅔ hyaline cartilage ≥50% |
| 5 | Loss of staining of all hyaline cartilage <50 |
| 6 | Loss of staining of all hyaline cartilage ≥50 |

Structural part

| | |
|---|---|
| 0 | Normal |
| 1 | Surface unevenness |
| 2 | cracks located at <50% of the surface layer |
| 3 | cracks located at ≥50% of the surface layer |
| 4 | Erosion of upper ⅓ hyaline cartilage <50% |
| 5 | Erosion of upper ⅓ hyaline cartilage ≥50% |
| 6 | Erosion of upper ⅔ hyaline cartilage <50% |
| 7 | Erosion of upper ⅔ hyaline cartilage ≥50% |
| 8 | Full depth injury in hyaline cartilage <50% |
| 9 | Full depth injury in hyaline cartilage ≥50% |
| 10 | Full depth erosion of subchondral bone, transparency, and |
| | calcification of cartilage <50% |
| 11 | Full depth erosion of subchondral bone, transparency, and |
| | calcification of cartilage ≥50% |

Chondrocyte density

| | |
|---|---|
| 0 | No reduction in the number of cells |
| 1 | Focal reduction in the number of cells |
| 2 | Multifocal reduction of cells |
| 3 | Multifocal fusion reduction of cells |
| 4 | Diffuse reduction of cells |

Particle aggregation formation

| | |
|---|---|
| 0 | Normal |
| 1 | <4 Colonies |
| 2 | 4 ≤ Cell colonies < 8 |
| 3 | Cell colonies ≥8 |

1.6 Data Statistical Analysis

The measurement was expressed as mean±standard deviation, and all data statistics were conducted using SPS S13.0 statistical software. Homogeneity of variance test was conducted for all data. One-way ANOVA was conducted for data with homogeneity of variance (P>0.05). LSD multiple comparisons analysis was conducted for data with differences (P≤0.05), with P≤0.05 indicating statistical differences; Kruskal Wallis non parametric testing was performed for data with uneven variances (P≤0.05), Mann Whitney pairwise comparison was performed for data with differences (P≤0.05), with P≤0.05 indicating statistical differences.

### 2 Results

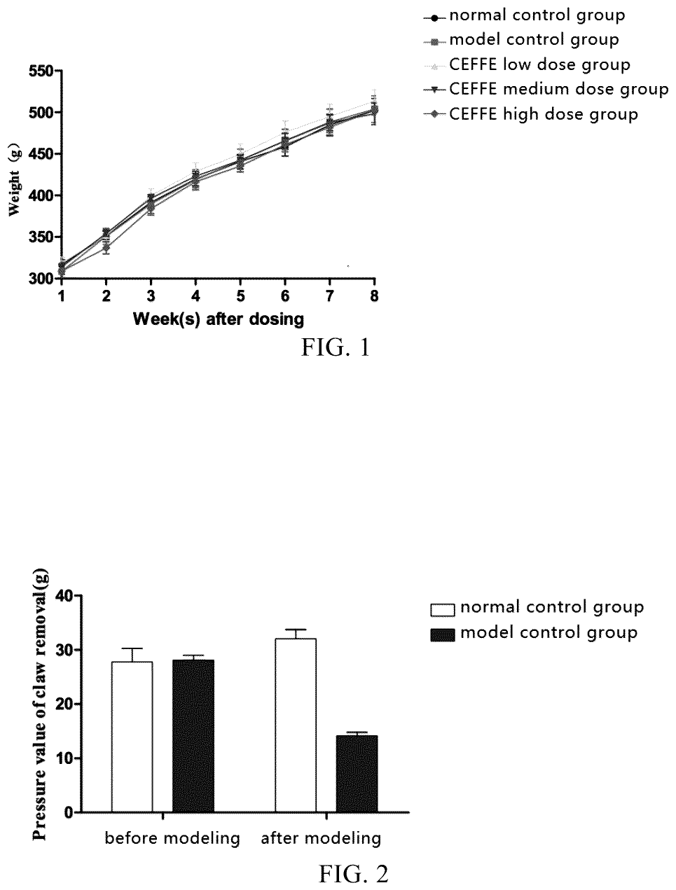

2.1 CEFFE Treatment has No Significant Changes in the General Situation of Model Rats After administration, no death or near death was observed in each group of animals. At the end point of the experiment, when euthanasia occurred (Day 54), there were no significant abnormalities observed during gross observation. The weight of each group of animals was similar before administration; after administration, the weight of each group of animals increased over time, and the weight at each time point was similar. The changes in weight in each group are shown in FIG. 1.

2.2 CEFFE Treatment Significantly Increases the Mechanical Pain Threshold in Model Rats

The changes in pressure value of claw removal before and after animal modeling are shown in FIG. 2. It can be seen from FIG. 2 that, before modeling, the pressure value of claw removal of rats in the modeling group and the normal control group were similar (28.1±5.0 vs 27.8±6.2 g, P>0.05). After one week of MIA modeling, the pressure value of claw removal of the modeling group was significantly lower than that of the normal control group (14.2±3.7 vs 32.1±4.1 g, P<0.001), indicating that MIA successfully induced osteoarthritis model in SD rats.

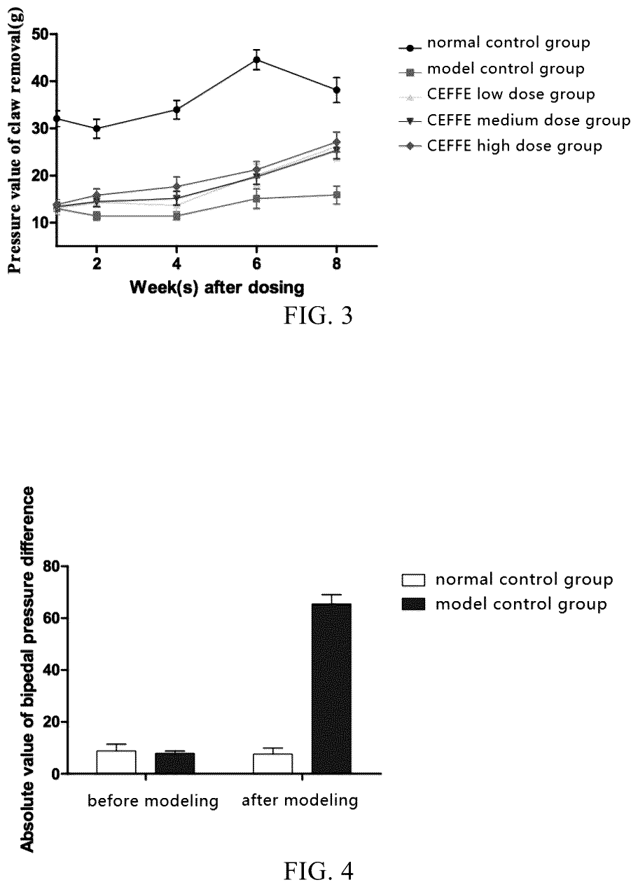

The pressure value of claw removal of arthritis model rats in each group before and after dosing are shown in FIG. 3. It can be seen from FIG. 3 that, before dosing, the pressure value of claw removal of rats in the model control group and CEFFE dose groups were similar, both significantly lower than those of the normal control group (P<0.001). During the experiment, the pressure value of claw removal of rats in the normal control group fluctuated between 30.0±4.9 and 44.5±5.2 g. After dosing, the fluctuation of pressure value of claw removal of the model control group was relatively small, significantly lower than that of the normal control group at each time points (P<0.001). The pressure value of claw removal of the CEFFE low dose group were similar to those of the model control group at each time points except for 1 week after the fourth dose (i.e., week 8), which was significantly higher than that of the model control group (26.2±7.5 vs. 15.9±4.7 g, P<0.01). The pressure value of claw removal of the CEFFE medium dose group were similar to those of the model control group at each time points except for 1 week after the fourth dose (i.e., week 8), which was significantly higher than that of the model control group (25.3±4.3 vs. 15.9±4.7 g, P<0.01). The pressure value of claw removal of the CEFFE high dose group was significantly higher than that of the model control group (P<0.05˜P<0.01) from one week after the first administration (i.e. the second week) to the experimental endpoint (one week after the fourth administration). The trend of changes in pressure value of claw removal of each group of animals before and after dosing is shown in FIG. 3.

Therefore, it can be seen from FIG. 3 that CEFFE has excellent therapeutic effects on the pain of osteoarthritis.

2.3 CEFFE Treatment Significantly Reduces Differences in Weight Distribution in the Hind Limbs and Alleviates Symptoms in Model Rats

The absolute value of bipedal pressure difference before and after modeling is shown in FIG. 4. It can be seen from FIG. 4 that, before modeling, the absolute value of bipedal pressure difference of the modeling group and that of the normal control group were similar (8±vs 9±7 g, P>0.05). One week after MIA modeling, the absolute value of bipedal pressure difference of the modeling group was significantly higher than that of the normal control group (66±19 vs 8±5 g, P≤0.001), indicating that MIA successfully induced an osteoarthritis model. The changes in absolute value of bipedal pressure difference before and after animal modeling are shown in FIG. 4.

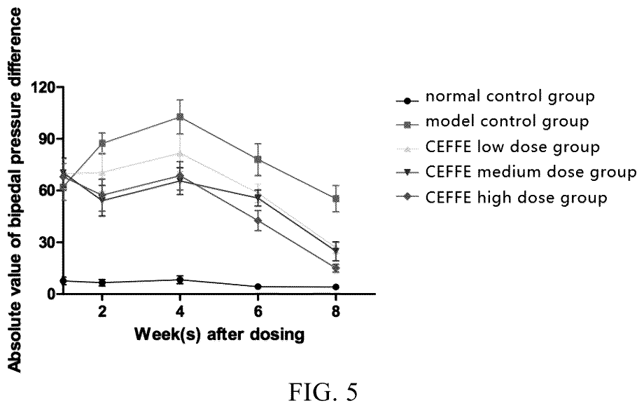

Before dosing, the absolute value of bipedal pressure difference of the model control group and that of the CEFFE groups were similar and significantly higher than those of the normal control group (P≤0.001). The absolute value of bipedal pressure difference of each group of arthritis model rats before and after dosing are shown in FIG. 5. It can be seen from FIG. 5 that, during the experimental administration process, the absolute value of bipedal pressure difference of the normal control group fluctuated between 4±2 and 8±6 g. After dosing, the absolute value of bipedal pressure difference at each time point of the model control group were significantly higher than those of the normal control group (P≤0.01˜P≤0.001). The absolute value of bipedal pressure difference of the CEFFE low dose group were similar to those of the model control group at each time points except for 1 week after the fourth dose (i.e., week 8), which was significantly lower than that of the model control group (27±8 vs. 55±19 g, P≤0.05). The absolute value of bipedal pressure difference of the CEFFE medium dose group were significantly lower than those of the model control group at each time points except for 1 week after the third dose (i.e., week 6), which was similar to that of the model control group. The absolute value of bipedal pressure difference of the CEFFE high dose group was significantly lower than that of the model control group (P≤0.05˜P≤0.01) from one week after the first administration (i.e. the second week) to the experimental endpoint (one week after the fourth administration).

The absolute value of bipedal pressure difference reflects joint weight-bearing, and the smaller the difference, the closer the weight-bearing is to normal. Bipedal pressure difference can definitely reflect the improvement of comprehensive symptoms in treating arthritis, including joint pain and movement disorder, etc. It can be seen from FIG. 5 that CEFFE can improve arthritis and the symptoms such as joint pain and movement disorder.

2.4 CEFFE Treatment Effectively Improves the Degree of Osteoarthritis in Model Rats

Under the microscope, slight to moderate articular cartilage fibrosis, mild to severe reduction in the number of articular chondrocytes, mild articular chondrocyte hyperplasia/degeneration, and slight to mild articular chondrocytes degeneration/necrosis/erosion were observed in the knee joints of the model control group animals. The above pathological changes are typical lesions of osteoarthritis, indicating successful modeling.

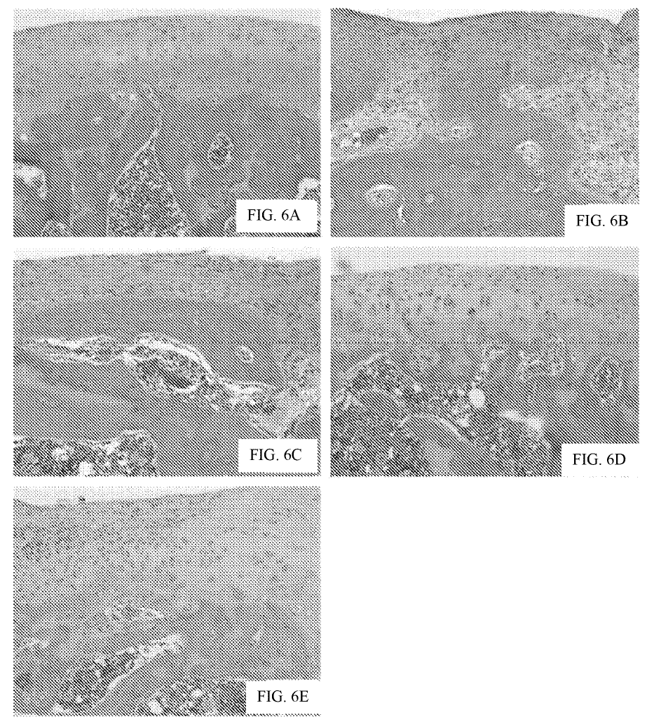

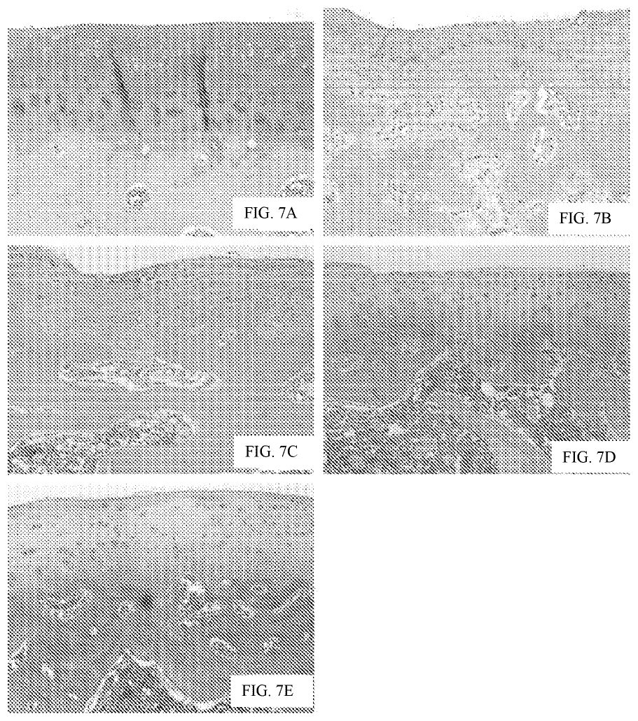

Slight to moderate articular cartilage fibrosis, mild to moderate reduction in the number of articular chondrocytes, slight to mild articular chondrocyte hyperplasia/degeneration, and slight to mild articular chondrocytes degeneration/necrosis/erosion can be observed in the knee joints of animals in the CEFFE low, medium, and high dose groups. Compared with the model control group, the incidence and/or degree of the above lesions in the CEFFE middle and high dose groups decreased, indicating that the CEFFE middle and high dose can improve the degree of lesion of MIA induced osteoarthritis in rats. The Safranin O-fast green staining results showed that, compared with the model control group, the score of Safranin O-fast green staining results in the CEFFE middle and high dose groups decreased. The HE staining and Safranin O-fast green staining results in different groups of rats are shown in FIGS. 6A-6E and FIGS. 7A-7E, respectively.

The histological results of HE staining in different groups of rats are as follows:

FIG. 6A. Normal control group (without any treatment), euthanized on the 54th day of the experiment (Day54), and there is no significant abnormality in the cartilage tissue of the knee joint (lower femur).

FIG. 6B. Model control group (physiological saline was injected to rats with osteoarthritis induced by MIA), euthanized on the 54th day of the experiment (Day54), and moderate articular cartilage fibrosis and severe reduction in the number of articular chondrocytes were observed in the knee joint (lower femur).

FIG. 6C. CEFFE low dose group, CEFFE was intra-articular injected to rats with osteoarthritis induced by MIA, euthanized on the 54th day of the experiment (Day54), and moderate articular cartilage fibrosis and severe reduction in the number of articular chondrocytes were observed in the knee joint (lower femur).

FIG. 6D. CEFFE medium dose group, CEFFE was intra-articular injected to rats with osteoarthritis induced by MIA, euthanized on the 54th day of the experiment (Day54), and slight articular cartilage fibrosis and slight reduction in the number of articular chondrocytes were observed in the knee joint (lower femur).

FIG. 6E. CEFFE high dose group, CEFFE was intra-articular injected to rats with osteoarthritis induced by MIA, euthanized on the 54th day of the experiment (Day54), and slight articular cartilage fibrosis and slight reduction in the number of articular chondrocytes were observed in the knee joint (lower femur).

The intergroup summary table of main pathological changes of knee joint HE staining in different groups of rats are shown in Table 3:

TABLE 3
Intergroup Summary of Main Pathological Changes of
Knee Joint HE Staining in Different Groups of Rats

| Group | | | | |
|---|---|---|---|---|
| 1 | 2 | 3 | 4 | 5 |
| Dose(mg/animal) | | | | |
| 0 | 0 | >0.0375 | >0.075 | >0.15 |
| Number of animals detected | | | | |
| 6 | 6 | 6 | 6 | 6 |

Knee joint-lower femur

| | | | | | | |
|---|---|---|---|---|---|---|
| Articular cartilage | Slight | 0 | 1 | 0 | 4 | 4 |
| fibrosis | Mild | 0 | 0 | 2 | 2 | 2 |
| | Moderate | 0 | 5 | 4 | 0 | 0 |
| Reduction in the number | Slight | 0 | 0 | 0 | 2 | 2 |
| of articular chondrocytes | Mild | 0 | 0 | 1 | 4 | 4 |
| | Moderate | 0 | 4 | 2 | 0 | 0 |
| | Severe | 0 | 2 | 3 | 0 | 0 |
| Articular chondrocytes | Mild | 0 | 1 | 0 | 0 | 0 |
| degeneration/necrosis | | | | | | |

Knee joint-upper tibia

| | | | | | | |
|---|---|---|---|---|---|---|
| Articular cartilage | Slight | 0 | 0 | 1 | 2 | 3 |
| fibrosis | Mild | 0 | 4 | 5 | 3 | 2 |
| | Moderate | 0 | 2 | 0 | 0 | 1 |
| Reduction in the number | Slight | 0 | 0 | 0 | 1 | 1 |
| of articular chondrocytes | Mild | 0 | 1 | 3 | 5 | 4 |
| | Moderate | 0 | 5 | 3 | 0 | 1 |
| Articular chondrocytes | Slight | 0 | 0 | 2 | 3 | 4 |
| hyperplasia/degeneration | Mild | 0 | 6 | 3 | 2 | 1 |

Knee joint-upper tibia

| | | | | | | |
|---|---|---|---|---|---|---|
| Articular cartilage | Slight | 0 | 0 | 1 | 2 | 3 |
| fibrosis | Mild | 0 | 4 | 5 | 3 | 2 |
| | Moderate | 0 | 2 | 0 | 0 | 1 |
| Reduction in the number | Slight | 0 | 0 | 0 | 1 | 1 |
| of articular chondrocytes | Mild | 0 | 1 | 3 | 5 | 4 |
| | Moderate | 0 | 5 | 3 | 0 | 1 |
| Articular chondrocytes | Slight | 0 | 0 | 2 | 3 | 4 |
| hyperplasia/degeneration | Mild | 0 | 6 | 3 | 2 | 1 |
| Articular chondrocytes | Slight | 0 | 3 | 4 | 4 | 2 |
| necrosis/erosion | Mild | 0 | 3 | 1 | 2 | 2 |

The histological results of Safranin O-fast green staining in different groups of rats are as follows:

FIG. 7A. Normal control group (without any treatment), euthanized on the 54th day of the experiment (D54), and there is no significant abnormality in the cartilage tissue of the knee joint (lower femur).

FIG. 7B. Model control group (physiological saline was injected to rats with osteoarthritis induced by MIA), euthanized on the 54th day of the experiment (D54), and moderate articular cartilage fibrosis and severe reduction in the number of articular chondrocytes were observed in the knee joint (lower femur).

FIG. 7C. CEFFE low dose group, CEFFE was intra-articular injected to rats with osteoarthritis induced by MIA, euthanized on the 54th day of the experiment (D54), and moderate articular cartilage fibrosis and severe reduction in the number of articular chondrocytes were observed in the knee joint (lower femur).

FIG. 7D. CEFFE medium dose group, CEFFE was intra-articular injected to rats with osteoarthritis induced by MIA, euthanized on the 54th day of the experiment (D54), and slight articular cartilage fibrosis and slight reduction in the number of articular chondrocytes were observed in the knee joint (lower femur).

FIG. 7E. CEFFE high dose group, CEFFE was intra-articular injected to rats with osteoarthritis induced by MIA, euthanized on the 54th day of the experiment (D54), and slight articular cartilage fibrosis and slight reduction in the number of articular chondrocytes were observed in the knee joint (lower femur).

The intergroup summary table of main pathological changes of knee joint safranin O-fast green staining in different groups of rats are shown in Table 4:

TABLE 4
Intergroup Summary of Main Pathological Changes of Knee Joint
safranin O-fast green Staining in Different Groups of Rats

| Group | | | | |
|---|---|---|---|---|
| 1 | 2 | 3 | 4 | 5 |
| Dose(mg/animal) | | | | |
| 0 | 0 | >0.0375 | >0.075 | >0.15 |
| Number of animals detected | | | | |
| 6 | 6 | 6 | 6 | 6 |

Knee joint-lower femur

| | | | | | | |
|---|---|---|---|---|---|---|
| Safranin O-fast green | 0 | 0 | 0 | 0 | 0 | 0 |
| staining score | 1 | 0 | 0 | 0 | 0 | 0 |
| | 2 | 0 | 0 | 0 | 0 | 0 |
| | 3 | 0 | 0 | 0 | 1 | 0 |
| | 4 | 0 | 0 | 0 | 1 | 2 |
| | 5 | 0 | 2 | 1 | 2 | 1 |
| | 6 | 0 | 4 | 5 | 2 | 3 |
| | Average | 0 | 5.7 | 5.8 | 4.8 | 5.2 |
| Structural part score | 0 | 0 | 0 | 0 | 0 | 0 |
| | 1 | 0 | 0 | 0 | 0 | 0 |
| | 2 | 0 | 0 | 0 | 0 | 0 |
| | 3 | 0 | 0 | 0 | 0 | 0 |
| | 4 | 0 | 0 | 0 | 0 | 0 |
| | 5 | 0 | 0 | 0 | 0 | 0 |
| | 6 | 0 | 0 | 0 | 1 | 0 |
| | 7 | 0 | 0 | 0 | 0 | 1 |
| | 8 | 0 | 0 | 0 | 1 | 2 |
| | 9 | 0 | 3 | 3 | 3 | 2 |
| | 10 | 0 | 1 | 2 | 1 | 1 |
| | 11 | 0 | 2 | 1 | 0 | 0 |
| | Average | 0 | 9.8 | 9.7 | 8.5 | 8.5 |
| Chondrocyte density | 0 | 0 | 0 | 0 | 0 | 0 |
| score | 1 | 0 | 0 | 0 | 0 | 0 |
| | 2 | 0 | 0 | 0 | 1 | 0 |
| | 3 | 0 | 0 | 1 | 4 | 4 |
| | 4 | 0 | 6 | 5 | 1 | 2 |
| | Average | 0 | 4 | 3.8 | 3.0 | 3.3 |
| Particle aggregation | 0 | 0 | 0 | 0 | 0 | 0 |
| formation score | 1 | 0 | 0 | 0 | 0 | 0 |
| | 2 | 0 | 0 | 0 | 0 | 0 |
| | 3 | 0 | 0 | 0 | 0 | 0 |
| | Average | 0 | 0 | 0 | 0 | 0 |

Knee joint-upper tibia

| | | | | | | |
|---|---|---|---|---|---|---|
| Safranin O-fast green | 0 | 0 | 0 | 0 | 0 | 0 |
| staining score | 1 | 0 | 0 | 0 | 0 | 0 |
| | 2 | 0 | 0 | 0 | 0 | 0 |
| | 3 | 0 | 0 | 0 | 0 | 0 |
| | 4 | 0 | 1 | 0 | 0 | 0 |
| | 5 | 0 | 1 | 2 | 6 | 3 |
| | 6 | 0 | 4 | 4 | 0 | 3 |
| | Average | 0 | 5.6 | 5.7 | 5 | 5.5 |
| Structural part score | 0 | 0 | 0 | 0 | 0 | 0 |
| | 1 | 0 | 0 | 0 | 0 | 0 |
| | 2 | 0 | 0 | 0 | 0 | 0 |
| | 3 | 0 | 0 | 0 | 0 | 0 |
| | 4 | 0 | 0 | 0 | 0 | 0 |
| | 5 | 0 | 0 | 0 | 0 | 0 |
| | 6 | 0 | 0 | 0 | 0 | 0 |
| | 7 | 0 | 1 | 0 | 0 | 0 |
| | 8 | 0 | 1 | 2 | 5 | 4 |
| | 9 | 0 | 2 | 2 | 0 | 2 |
| | 10 | 0 | 0 | 1 | 1 | |
| | 11 | 0 | 2 | 1 | 0 | 0 |
| | Average | 0 | 9.2 | 9.2 | 8.3 | 8.3 |
| Chondrocyte density | 0 | 0 | 0 | 0 | 0 | 0 |
| score | 1 | 0 | 0 | 0 | 0 | 0 |
| | 2 | 0 | 0 | 0 | 0 | 0 |
| | 3 | 0 | 2 | 2 | 6 | 3 |
| | 4 | 0 | 4 | 4 | 0 | 3 |
| | Average | 0 | 3.7 | 3.7 | 3 | 3.5 |
| Particle aggregation | 0 | 0 | 0 | 0 | 0 | 0 |
| formation score | 1 | 0 | 1 | 0 | 0 | 0 |
| | 2 | 0 | 1 | 0 | 0 | 0 |
| | 3 | 0 | 3 | 0 | 0 | 0 |
| | Average | 0 | 2 | 0 | 0 | 0 |

### 3 Conclusion

This study used CEFFE intra-articular injection to treat MIA induced osteoarthritis model rats. Behavioral studies have shown that CEFFE treatment can effectively increase the mechanical pain threshold, alleviate mechanical pain, reduce bipedal pressure difference, alleviate weight-bearing abnormalities in the hind limbs caused by osteoarthritis, and alleviate symptoms of osteoarthritis in model rats. Histopathology results also confirm that CEFFE treatment can effectively reduce the degree of lesion of osteoarthritis and reduce the destruction of cartilage tissue. In summary, CEFFE has excellent therapeutic effects on osteoarthritis.

All the documents cited herein are incorporated into the invention as reference, as if each of them is individually incorporated. Further, it would be understood that, in light of the above-described teaching of the invention, the skilled in the art could make various changes or modifications to the invention, and these equivalents are still in the scope of the invention defined by the appended claims of the application.

1. A method for (i) preventing and/or treating arthritis; (ii) preventing and/or treating pain; (iii) movement disorder, comprising the step of: administering a cell-free fat extract or a composition or preparation containing the cell-free fat extract to a subject in need thereof.

2. The method according to claim 1, wherein the arthritis includes osteoarthritis.

3. The method according to claim 2, wherein the osteoarthritis includes degenerative osteoarthritis.

4. The method according to claim 2, wherein the osteoarthritis is selected from the group consisting of cervical osteoarthritis, lumbar osteoarthritis, knee osteoarthritis, hip osteoarthritis, and combinations thereof.

5. The method according to claim 1, wherein the pain includes arthritis pain; and/or the movement disorder include the movement disorder caused by arthritis.

6. The method according to claim 1, wherein the cell-free fat extract contains one or more components selected from the group consisting of IGF-1, BDNF, GDNF, TGF-β1, HGF, bFGF, VEGF, TGF-β1, HGF, PDGF, EGF, NT-3, GH, G-CSF, and the combinations thereof.

7. The method according to claim 6, wherein the cell-free fat extract comprises one or more features selected from the group consisting of:
in the cell-free fat extract, the concentration of IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml;
in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml;
in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml;
in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml;
in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml;
in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml;
in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml;
in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

8. The method according to claim 6, wherein the cell-free fat extract comprises one or more features selected from the group consisting of:
the weight ratio of IGF-1 to VEGF is 20-100:1, preferably 30-70:1, more preferably 40-60:1, and most preferably 45-55:1;
the weight ratio of BDNF to VEGF is 2-20:1, preferably 4-15:1, more preferably 6-12:1, and most preferably 8-9.5:1;
the weight ratio of GDNF to VEGF is 2-20:1, preferably 4-15:1, more preferably 6-12:1, and most preferably 8.5-9.5:1;
the weight ratio of bFGF to VEGF is 0.2-8:1, preferably 0.5-5:1, more preferably 0.6-2:1, more preferably 0.8-1.6:1, and most preferably 1-1.5:1;
the weight ratio of TGF-β1 to VEGF is 1-20:1, preferably 1-15:1, more preferably 1-10:1, more preferably 2-8:1, more preferably 4-6:1;
the weight ratio of HGF to VEGF is 1-20:1, preferably 1-15:1, more preferably 1-10:1, more preferably 2-8:1, more preferably 4-5.5:1; and/or
the weight ratio of PDGF to VEGF is 0.1-3:1, preferably 0.2-2:1, more preferably 0.4-1.5:1, and most preferably 0.7-1.2:1.

9. The method according to claim 1, wherein the dosage form of the composition or preparation is an oral preparation, an external preparation, or an injectable preparation.

10. The method according to claim 1, wherein the dosage form of the composition or preparation is powder, granule, capsule, injection, tincture, oral solution, tablet or lozenge.

11. The method according to claim 1, wherein the cell-free fat extract contains no cell and no lipid droplet.

12. The method according to claim 1, wherein the cell-free fat extract is a naturally-obtained nano-fat extract without added ingredients.

13. The method according to claim 1, wherein the cell-free fat extract is prepared by the following method:
(1) providing a fat tissue raw material, shredding the fat tissue raw material, and rinsing (e.g., with physiological saline), thereby obtaining a rinsed fat tissue;
(2) centrifuging the rinsed fat tissue to obtain a layered mixture;
(3) discharging the oil at the upper layer and the liquid at the bottom layer from the layered mixture and collecting the intermediate layer (that is, the fat layer containing fat cells);
(4) subjecting the intermediate layer to emulsification to obtain a emulsified fat mixture (also called nano-fat);
(5) centrifuging the emulsified fat mixture to obtain an intermediate liquid layer, which is a primary fat extract; and
(6) subjecting the primary fat extract to filtration and sterilization to obtain the cell-free fat extract.

14. A cell-free fat extract, wherein the cell-free fat extract is prepared by the following method:
(1) providing a fat tissue raw material, shredding the fat tissue raw material, and rinsing (e.g., with physiological saline), thereby obtaining a rinsed fat tissue;
(2) centrifuging the rinsed fat tissue to obtain a layered mixture;
(3) discharging the oil at the upper layer and the liquid at the bottom layer from the layered mixture and collecting the intermediate layer (that is, the fat layer containing fat cells);
(4) subjecting the intermediate layer to emulsification to obtain a emulsified fat mixture (also called nano-fat);
(5) centrifuging the emulsified fat mixture to obtain an intermediate liquid layer, which is a primary fat extract; and
(6) subjecting the primary fat extract to filtration and sterilization to obtain the cell-free fat extract.

15. A composition or preparation comprising (a) a cell-free fat extract according to claim 14; and (b) pharmaceutically, food, nutraceutical, or dietary acceptable carriers or excipients.

16. The method according to claim 1, wherein the prevention and/or treatment of arthritis is carried out through one or more means selected from the group consisting of:
(a) inhibiting fibrosis of articular cartilage; and/or
(b) improving the number of articular chondrocytes.

17. The method according to claim 12, wherein the expression of “contain no lipid droplet” means that in the cell-free fat extract, the volume of oil droplets accounts for less than 1% of the total liquid;
the expression of “contain no cell” means that the average number of cells in 1 mL of the cell-free fat extract is ≤1.
